(19) United States
(12) Patent Application Publication
(10) Pub. No.: US 2016/0287816 A1
(43) Pub. Date: Oct. 6, 2016

(54) Vaporizer

(71) Applicant: Cloud V Enterprises, Glendale, CA (US)

(72) Inventor: Vahan Eksouzian, Glendale, CA (US)

(73) Assignee: Cloud V Enterprises, Glendale, CA (US)

(21) Appl. No.: 14/672,232

(22) Filed: Mar. 30, 2015

(51) Int. Cl. A61M 11/04; A61M 15/00

(52) U.S. Cl. CPC A61M 11/042; A61M 15/0021; A61M 2205/8206

(57) ABSTRACT

A vaporizer comprising a mouthpiece, atomizer and battery. The atomizer preferably includes an upper chamber and a lower chamber disposed directly below the upper chamber. The upper chamber is preferably the shape of part of an oval cone and the lower chamber is preferably the shape of an oval cylinder. A heat source is disposed in the lower chamber, preferably running lengthwise along the longest diameter of the oval cross-section of the lower chamber. The atomizer is detachable connectable to the battery's housing, facilitating an electrical connection between the atomizer and the battery.

### BACKGROUND OF THE INVENTION

1. Field of the Invention

The present invention relates generally to the field of vaporizers used to heat solids, liquids and suspensions, such as waxes and oils, for inhalation by a user.

2. Prior Art

Vaporizers including electronic cigarettes, have become increasingly popular over the past ten years. As consumers have become more aware of the health consequences of inhaling smoke produced by combusting tobacco arid other substances, vaporizers have become a healthier alternative to cigarettes, cigars, pipes and oilier smoking implements. While some vaporizers are large and bulky—sometimes intended to mimic the aesthetic look of a hookah—most vaporizers are small enough to fit into a user's pocket or purse for convenience.

The typical configuration of a compact vaporizer includes a battery electrically connected to an atomizer housed within the vaporizer. The solid, liquid or suspension material sought to be vaporized is placed in the atomizer, or alternatively placed in a separate housing connected to the atomizer by a wick. The atomizer includes one or more heating coils electrically connected to a battery. When activated, the heating coil heats the substance in contact with the heating coil and vaporizes that substance so that the user may inhale the resulting vapor.

Many compact vaporizers are cylindrical in shape. The corresponding atomizers disposed inside the atomizers are also cylindrical in shape with a circular cross-section. An example of such an atomizer and vaporizer is described in U.S. Pat. No. 8,851,068 issued to Cohen et al. The atomizers typically have a single heating coil running from one side of the circular cross-section to the opposite side. Other compact vaporizers have an oval cross-section, yet still the corresponding atomizers have a circular cross-section The circular cross-section, particularly when employed in a cylindrical vaporizer, has Certain advantages most notably that it may be removably attached to the vaporizer housing unit with a conventional thread connection, much like a bolt and nut. However, the circular cross-section also has significant disadvantages. For example, with a circular cross-section a significant amount of the inserted material does not contact the heating coil and may accumulate on or near the walls of the vaporizer furthest from the heating coil. This is particularly problematic where the walls of the atomizer are of a material that may interact chemically with the substance loaded into the atomizer. Regardless of the atomizer housing material, it is undesirable to have unused material remain inside the atomizer, particularly if one or more users wish to use the vaporizer to inhale different substances.

The conventional atomizer utilized in compact vaporizers is best understood with reference to FIGS. 1, 2 and 3, which depict such atomizers. Conventional atomizers can be conceptualized m two hollow cylinders disposed adjacent to one another. The smaller cylinder houses a metal heating coil 2, and is defined by a lower chamber wall 4. The lower chamber wall 4 is typically a metallic material. The upper cylinder is defined by an upper chamber wall 6. The upper chamber wall 6 may also be a metallic material, though some atomizers have employed upper chamber walls made of plastic, presumably due its low cost and good insulating properties. Plastic proved undesirable and unsuitable as a material for the upper chamber wall 6 because the heat emitted by the heating coil 2 occasionally caused the plastic to melt or combust, causing the user to inadvertently inhale fumes from burnt plastic or resulting in the heating coil 2 being coated with melted plastic and thus disabling the heating coil 2. The inner diameter of the upper chamber wall 6 of conventional atomizers for use in compact vaporizers are generally less than one inch, and the diameter of the lower chamber wall 4 is even smaller.

Liquid and suspension materials are loaded by the user into the atomizer. Liquids, such as e-liquid containing nicotine, may be dropped into the smaller cylinder, but it is likely, due to the small size of the smaller cylinder in particular, that some of the liquid will be caught in the larger cylinder and not travel down to the smaller cylinder, particularly in the corners 9 of the upper cylinder. Suspensions and relatively high viscosity fluids, such as wax and oil, are loaded into the atomizer using a “dabbing” tool. The suspension or high viscosity fluid is scraped off the dabbing tool onto the inner portion of the upper chamber wall 6. The suspension or high viscosity fluid may he then manually packed down into the lower chamber, or, depending on the properties of the suspension or high viscosity fluid, the user may activate the heating coil 2, and rely on gravity and the reduced viscosity of the fluid (caused by the increase in temperature) to cause the suspension or high viscosity fluid to fall into the smaller cylinder. With suspensions and high viscosity fluids it is even more likely that a significant amount of the substance sought to be vaporized will get trapped in the corners 9 of the upper cylinder, or the pockets 7 in the smaller cylinder furthest from the heating coil 2.

One solution to the problems discussed above may be the use of multiple beating coils disposed in various locations within the atomizer. An example of a vaporizer with multiple heating coils is described in U.S. Pat. No. 8,881,737 issued to collect et al. However, providing multiple heating coils is often impractical because of the additional manufacturing complexity and cost, as well as the desired small size of the vaporizer. Another solution may be to increase the amount of heat produced by the locating coil, by, for example, increasing the voltage and/or current from the battery to the heating coil. However, by increasing the heat produced by the heating coil, it becomes more likely that the loaded substance will combust, rather than just vaporize. Therefore, while increasing the heat produced by the heating coil, and thus increasing the temperature in all portions of the atomizer, might be m acceptable solution in certain applications, increasing tire temperature might cause certain substances to combust, thus eliminating the health benefits of vaporizing relative to smoking and also potentially damaging the vaporizer.

There exists a need far a compact vaporizer that includes an atomizer that cleanly and efficiently directs the substance sought to be vaporized toward a heating coil to minimize the proportion of the substance left unvaporized. The present invention, the preferred embodiment of which is described herein, meets this need, and further provides an atomizer that conveniently and reliably engages and disengages the vaporizer, and ensures no degradation or chemical reaction between the atomizer and the loaded substance.

### SUMMARY OF THE INVENTION

The present invention, or more accurately the preferred embodiment of the present invention, provides a compact vaporizer comprising an atomizer that maximizes contact between a heating coil and a substance loaded into the atomizer by the user. The shape of the vaporizer and the position of the heating coil minimize the amount of the loaded substance dial is left unvaporized. The atomizer is easily and conveniently attached to, and detached from the vaporizer housing unit. When the atomizer is attached to the vaporizer housing unit it is also in electrical contact with the battery. The atomizer includes at least one heating coil, and the inside wall of the atomizer is shaped so as to cause a liquid or suspension, such as wax, oil or e-juice, to be directed toward contact with, or close proximity to the heating coil. The heating coil, or alternatively a small heating dish, is likewise positioned in the atomizer to heat, and thus vaporize, the loaded liquid or suspension, particularly the portion of the loaded liquid or suspension that is in contact with, or close proximity to, the heating coil. Preferably, the inside wall of the atomizer is constructed from a material that will not degrade from at temperatures up to at least 400 degrees Fahrenheit, and will not degrade or otherwise react chemically with substances commonly vaporized, including wax, oil and e-juice.

### BRIEF DESCRIPTION OF THE DRAWINGS

FIG. 1 is a perspective view of an atomizer consistent with the prior art.

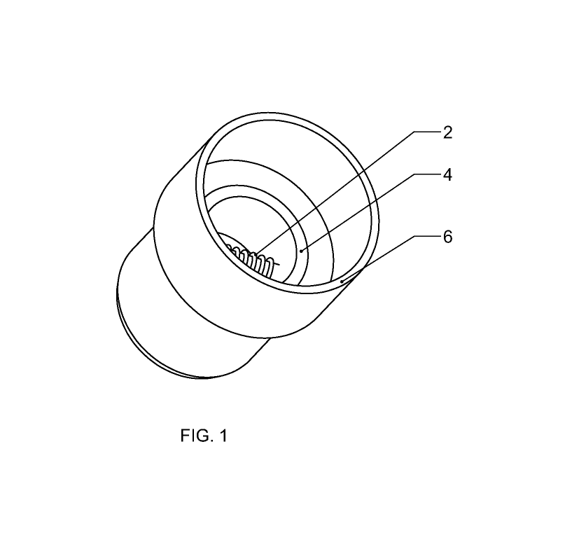

FIG. 2 is an overhead view of an atomizer consistent with the prior art.

FIG. 3 is a schematic cross-sectional view of an atomizer consistent with the prior art.

FIG. 4 is a perspective view of a compact vaporizer consistent with the preferred embodiment of the present invention.

FIG. 5 is a perspective view of an atomizer consistent with the preferred embodiment of the present invention present invention.

FIG. 6 is an overload view of an atomizer consistent with the preferred embodiment of the present invention present invention.

FIG. 7 is a schematic cross-sectional view of an atomizer consistent with the preferred embodiment of the present invention present invention.

### DETAILED DESCRIPTION OF THE PREFERRED EMBODIMENT OF THE INVENTION

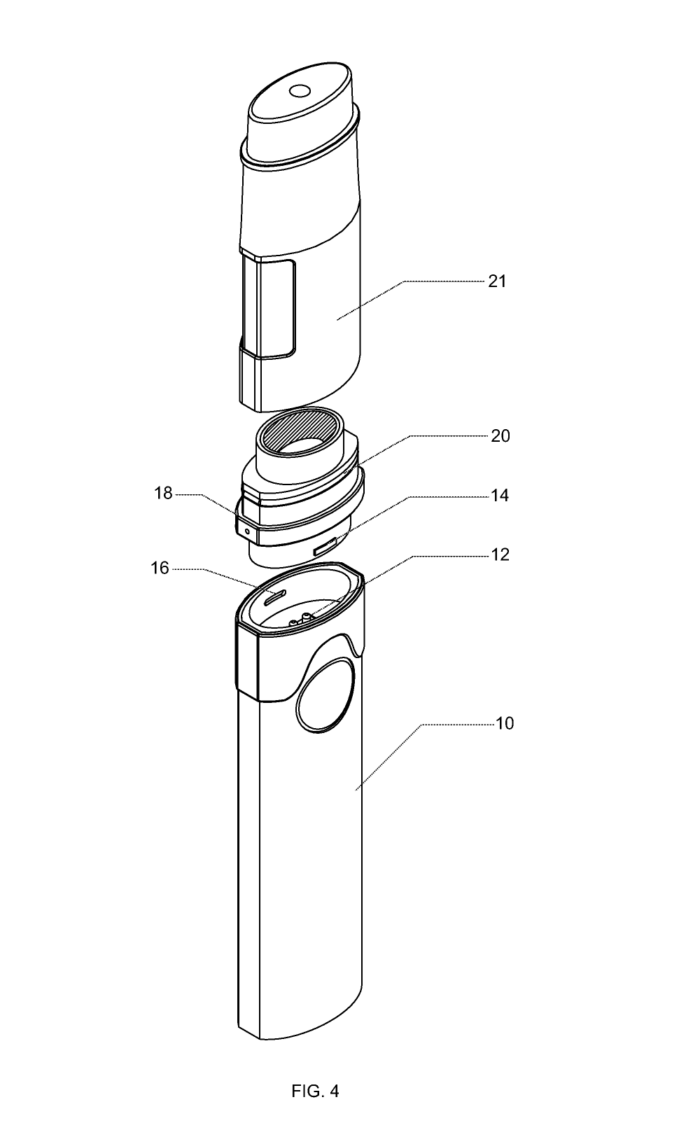

As depicted in FIG. 4, the prefer red embodiment compact vaporizer includes a battery housed within a battery casing 10. The battery includes two leads 12 (one positive and one negative) suited to connect electrically to an atomizer 18. The atomizer 18 includes at least one protrusion 14 configured to adapt to a recess 16 on the inside wall of the vaporizer. The protrusion 14 is preferably made of a flexible material. The protrusion 14 and recess 16 are sized and configured to enable a male-female connection between the atomizer and the battery easing 10 when the atomizer 18 is placed in electrical connection with the battery through leads 12. The atomizer 18 has a gasket 20 extending around the circumference of the atomizer 18. The gasket is preferably made of a flexible material, such as rubber or a flexible plastic, suitable for creating an airtight seal between the atomizer and mouthpiece 21. The gasket 20 and mouthpiece 21 are sized so as to allow easy attachment and removal of the mouthpiece 21 from the atomizer 18. In that way, a user may readily access the atomizer 18 to load a substance that the user desires to vaporize, then attach the mouthpiece 21 to allow the user to inhale the vaporized substance. The connection between the battery and atomizer that is secured by the interlocking of the protrusion 14 with the recess 16 allows the user to remove the mouthpiece 21 for access to the atomizer 18 without disconnecting the atomizer 18 from the battery housing 10 and leads 12. Those skilled in the art will recognize that the detachable connection between the lottery housing 10 and atomizer 18 may alternatively be accomplished with a protrusion on the battery housing and a recess in the atomizer. Upon loading a substance into the atomizer 18 and attaching the mouthpiece 21, the user may activate the battery, for example by pressing a button, to heat the substance in the atomizer 18, and inhale the vaporized substance through the opening at the top of the mouthpiece 21.

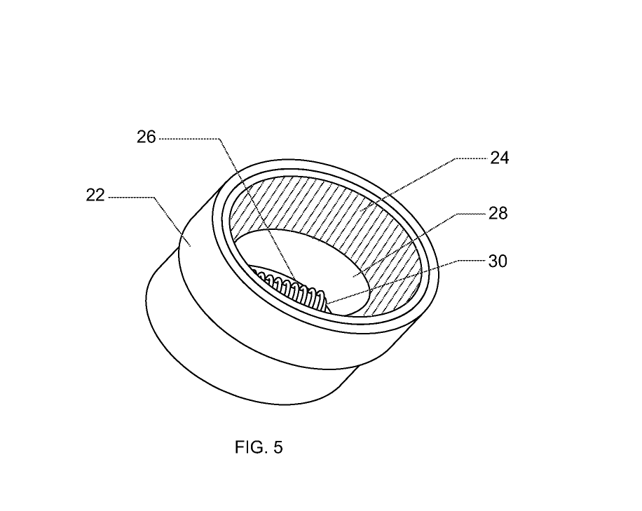

Turning to FIG. 5, the atomizer of the preferred embodiment includes an outer wall 22. The outer wall 22 may be any suitable material capable of withstanding heal and repeated changes in temperature, but is preferably metallic or ceramic. The atomizer also includes an upper-inner wall 24 and a lower-inner wall 28. As is best seen in the Figures discussed below, the cross sections of the upper-inner wall 24 and the tower-inner wall 28 are both oval in shape. The upper-inner wall 24 has a decreasing circumference as the upper-inner wall gets closer to the heating coil 26. The lower-inner wall 28 has a circumference equal to the smallest cross-sectional circumference of the upper-inner wall 24—i.e. where the upper-inner wall 24 is closest to the heating coil 26. The upper-inner wall 24 and lower inner wall 28 are both preferably made of a material that is a good conductor of heat, will not react chemically with substances commonly used in vaporizers, and can withstand high and repeatedly changing temperatures, such as ceramic. Optionally, a wick 30 is disposed inside of the heating coil to absorb the substance to be vaporized and bring it into close proximity of the heating coil 26. Those skilled in the art will understand that the wick 30 may be a fiber wick, a ceramic rod, or some other material suitable for withstanding temperatures of at least 500 degrees Fahrenheit.

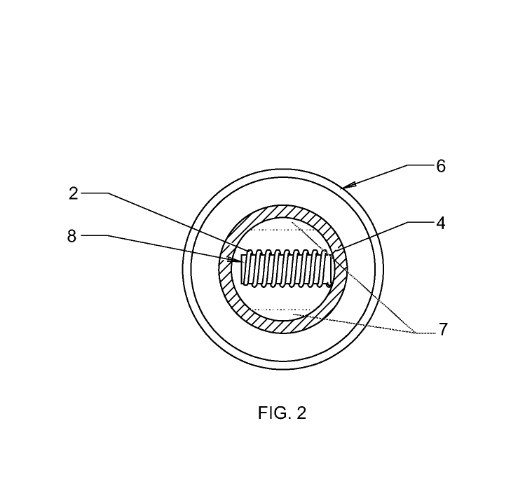

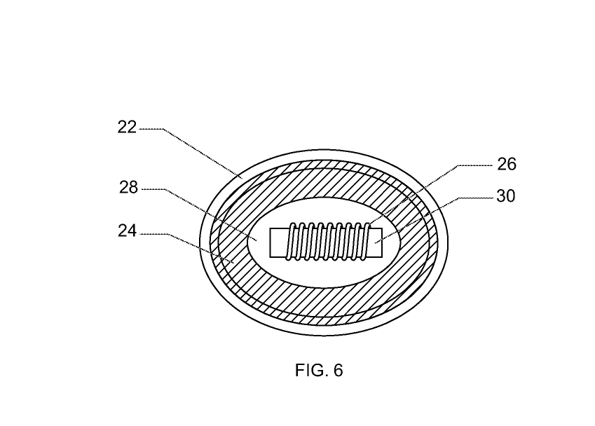

Turning to FIG. 6, the principal advantage of an oval-shaped atomizer over a similar circular-shaped atomizer is shown. Whereas with a circular-shaped atomizer, such as that shown in FIG. 2, there are pockets 7 where a substance may accumulate because it is too far from the heating coil 2, there are no such pockets with an oval-shaped atomizer, particularly where the lower-inner wall 28 has an oval cross section. Rather, with an oval shaped cross-section, the greatest distance between the lower-inner wall 28 to the heating coil 26 is less than that with a circular shaped cross-section.

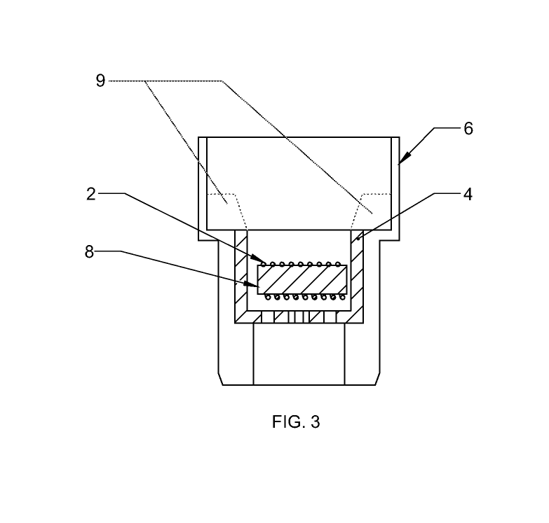

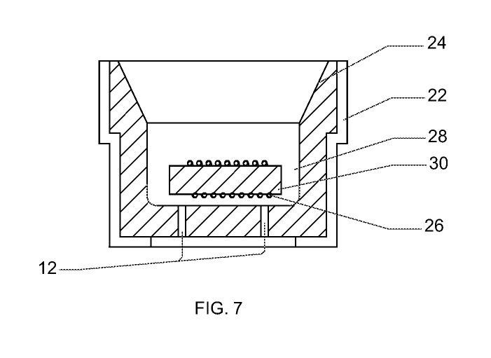

Turning to FIG. 7, some of the advantages of the configuration of the preferred embodiment of the present invention are shown. First, the conical shape of the upper-inner wall 24 facilitates easy loading at the top of atomizer 18, and directs all of the substance to the area war the heating coil 26 defined by the lower-inner wall 28, particularly due to the circumference of the upper-inner wall 24 being larger at its top and decreasing to equal the circumference of the lower-inner wall 28 at its bottom. This is in stark contrast to the conventional atomizer configuration shown in FIG. 3, where a loaded substance may accumulate unvaporized in the corners 9. With the upper-inner wall 24 of the preferred embodiment of the present invention, there are no comers where the substance may accumulate and go unvaporized. Similarly, the rounded corners at the bottom of the lower cylinder defined by the lower-inner wall 28 prevent the accumulation of unvaporized substance in those corners that might otherwise result with sharp corners. Through the configuration shown in FIG. 7, both gravity and the heat emitted from the heating coil 26 cause a substance scraped anywhere onto the upper-inner wall 24 to travel towards the heating coil 26 where it is vaporized such that it may be inhaled by a user.

While FIGS. 5 through 7 depict an atomizer with a heating coil 26 wrapped around a wick 30, it should he recognized that it may be advantageous to eliminate the wick 10. It may also be advantageous to replace Use heating coil 26 and wick 30 with a heated pan at the bottom of the atomizer 18. The heated pan may consist of any heat-conductive material(s), but preferably is ceramic where the pan comes in contact with the substance to he vaporized. This configuration provides even greater interchangeability of substances because there is no absorption of any substance in a porous wick such that a small quantity of the substance would remain and be vaporized in a subsequent use.

It is understood that many modifications and variations may be devised given the above description of the principles of the invention and a preferred embodiment of the invention. It is intended that all such modifications and variations be considered within the spirit and scope of this invention, as defused by the following claims.

The invention claimed is:

1. A vaporizer comprising;
a mouthpiece;
an electrical source; and
an atomizer electrically connectable with said electrical source comprising a heat source, wherein the inside of said atomizer has a consistently decreeing inner circumference front the top of said atomizer to a height lower than the top of said atomizer.

2. The vaporizer of claim 1 wherein said atomizer further comprises:
an upper chamber defined by an upper wall, wherein the circumference of said upper wall is consistently decreasing as said upper wall approaches said heat source; and
a lower chamber defined by a lower waif wherein said heat source is disposed in said lower chamber and the circumference of said lower wall is equal to the least circumference of said upper wall.

3. The vaporizer of claim 1, wherein said heat source is a heating coil.

4. The vaporizer of claim 3, wherein said lower chamber has a cross-section that is oval in shape and said heating coil runs longitudinally along the longest diameter of said oval-shaped cross-section.

5. The vaporizer of claim 1, wherein said heat source is a heated pan.

6. The vaporizer of claim 1, wherein said electrical source is capable of being activated by a user pressing a button.

7. The vaporizer of claim 1, wherein said electrical source is housed in an electrical source casing, and said electrical source casing is detachably connectable to said atomizer by a protrusion of said atomizer interfacing with a recess in said electrical source casing.

8. An atomizer comprising:
an upper chamber defined by an upper wall, wherein the upper wall is conical in shape such that the inner circumference of the upper chamber is consistently decreasing from a top circumference to a bottom circumference;
a lower chamber defined by a lower wall and housing a heat source, wherein said lower chamber is cylindrical in shape and the inner circumference of the lower chamber is equal to said bottom circumference

9. The atomizer of claim 8, wherein said heat source is a heating coil.

10. The atomizer of claim 9 wherein said lower chamber has a cross-section that is oval in shape and said healing coil runs longitudinally along the longest diameter of said oval-shaped cross-section.

11. The atomizer of claim 8, wherein said heat source is a heated pan.

12. A vaporizer comprising:
a mouthpiece;
an electrical source; and
an atomizer electrically connectable with said electrical source comprising
an upper chamber defined by an upper wall; and
a lower chamber defined by a lower wall, wherein said lower chamber houses a heat source, and wherein said lower chamber has a cross-section that is oval in shape.

13. The vaporizer of claim 12, wherein said heat source is a heating coil that runs longitudinally along the longest diameter of said oval cross-section of said lower chamber.

14. The vaporizer of claim 12, wherein said upper chamber is conical.

15. The vaporizer of claim 14, wherein said upper chamber is an oval cone.

16. The vaporizer of claim 12, wherein said electrical source is housed in a battery housing and said atomizer is detachably connectable to said batten housing by the interface of a protrusion on said atomizer and a recess in said battery housing.

17. The vaporizer of claim 12, wherein said electrical source is housed in a battery housing and said atomizer at detachably connectable to said battery housing by the interlace of a protrusion on said battery housing and a recess in said atomizer.
